(19) United States
(12) Patent Application Publication
(10) Pub. No.: US 2022/0264910 A1
(43) Pub. Date: Aug. 25, 2022

(54) COMPOSITIONS COMPRISING AMINO ACIDS AND A FURTHER COMPONENT FOR THE SUPPLY OF AMINO ACIDS TO A MONOGASTRIC ANIMAL SUCH AS A HUMAN OR A PIG

(71) Applicant: VETAGRO INTERNATIONAL S.R.L., Reggio Emilia (RE) (IT)

(72) Inventor: Andrea PIVA, Reggio Emilia (RE) (IT)

(21) Appl. No.: 17/631,182

(22) PCT Filed: Jul. 31, 2020
(86) PCT No.: PCT/IB2020/057255

§ 371 Date: Jan. 28, 2022

(30) Foreign Application Priority Data
Jul. 31, 2019 (IT) 102019000013473

(51) Int. Cl. A23K 20/142; A23K 20/158; A23K 50/30; A23L 33/105; A23L 33/175; A61K 31/198; A61K 45/06; A61K 9/16

(52) U.S. Cl. CPC A23K 20/142; A23K 20/158; A23K 50/30; A23L 33/105; A23L 33/175; A61K 31/198; A61K 45/06; A61K 9/1617

(57) ABSTRACT

A composition comprising at least one amino acid, (ii) a controlled release lipid matrix, and optionally (iii) at least one acceptable pharmaceutical or food grade additive and/or excipient and a related method for the treatment of amino acid supply or protein deficiency in a monogastric subject, preferably a human subject or a pig, are disclosed as well as a feed or feed additive comprising the composition for a monogastric animal, preferably a pig.

The present invention relates to a composition, preferably in the solid form of granules, comprising at least one amino acid and a lipophilic matrix for use in a method for the treatment of an amino acid deficiency in a mammalian monogastric subject, such as a human subject or a pig.

According to a first embodiment (FR-I), the present invention relates to a composition comprising (i) a mixture comprising or, alternatively, consisting of (a) a first active component, such as (a1) at least one amino acid and/or (a2) at least one whey protein, and (b) a second active component, such as (b1) at least one protease, (b2) alpha-ketoglutaric acid, (b3) ornithine and a mixture thereof; wherein said composition further comprises (ii) a controlled release lipid matrix, and, optionally, (iii) at least one acceptable pharmaceutical or food grade additive and/or excipient; wherein said (ii) lipid matrix allows a gastroprotection and controlled release of said (i) mixture of active components (i.e. amino acids) into the intestine, ensuring a constant blood bioavailability within a period of time comprised in the range from 2 hours to 24 hours. According to a second embodiment (FR-II), the present invention relates to a composition comprising (i) a mixture comprising or, alternatively, consisting of (a) a first active component, such as (a1) at least one amino acid and/or (a2) at least one whey protein, and (b) a second active component, such as (b4) at least one phytocompound derivative (botanical) (i.e. thymol, carvacrol, eugenol, capsaicin, tannins, verbascoside and mixtures thereof); wherein said composition further comprises said (ii) controlled release lipid matrix, and, optionally, said (iii) at least one additive and/or excipient. Furthermore, the present invention relates to said composition (according to FRI or FRII) for use in a method for the treatment of amino acid supply or protein (or amino acid) deficiency and of diseases, symptoms and/or disorders deriving from said protein deficiency in a monogastric subject, preferably a human subject or a pig. Lastly, the present invention relates to the use of said composition, comprising (i) and (ii) and, optionally, (iii), for the preparation of a feed or feed additive for a monogastric animal, preferably a pig.

The development and maintenance of skeletal muscle mass are determined by the sum of muscle protein synthesis processes (in short MPS, process of hypertrophy) and muscle protein breakdown (in short MPB, atrophy process).

In particular, in humans the preservation and development of muscle mass, determined by the homeostatic balance between MPS and MPB, are essential elements for the maintenance of metabolic health and independent locomotion, i.e. generally, of a better quality of life. This balance between MPS and MPB can be disturbed by various factors such as chronic diseases, muscle disuse and ageing. As a matter of fact, loss of muscle mass and strength (sarcopaenia) is one of the factors most responsible for increased mortality, morbidity, and reduced quality of life in the elderly. It has been observed that intravenous administration of amino acids (AA) in human volunteer subjects, promoting hyperaminoacidemia and hyperinsulinemia, stimulates MPS. However, muscle protein synthesis is a process considered “saturable”, hence the amino acid composition of the protein source and the amount of essential amino acids (EAAs) supplied through the diet is crucial. As concerns humans, there are nine EAAs, i.e. the AAs that the organism is not able to synthesise de novo and must therefore be acquired through the diet: histidine, isoleucine, leucine, lysine, methionine, phenylalanine, threonine, tryptophan, valine. As concerns pigs, this list of EAAs also includes: arginine, cysteine and tyrosine. Out of these nine human EAAs, recent studies have shown that leucine, one of the branched-chain AAs (BCAAs), plays a crucial role in MPS through the activation of signal cascades in the mTORC1 pathway, both in humans and in pigs. As a matter of fact, this AA was identified as the main anabolic signal among the different AAs. During the postprandial phase (1-4 h after meal) MPS is high, resulting in a positive muscle protein balance, while MPS rate is lower in the fasting phase and protein balance is negative. Furthermore, the blood concentration of EAAs has been shown to regulate the rate of protein synthesis in the muscle at rest and after exercise.

In monogastric animals, amino acid supply through feeding plays a fundamental role in their growth. To date, in order to satisfy the wide market demands, the rearing of monogastric animals, such as pigs, is carried out on a large scale and requires the consumption of considerable amounts of feed to ensure an appropriate supply of amino acids to said animals. If the content of amino acids or proteins in the feed of monogastric animals is not well balanced, both quality and quantity-wise, excess amino acids are not used for metabolic purposes and they are excreted by the animal, causing a negative environmental impact due to high nitrogen excretion. Furthermore, undigested excess protein in the gut can lead to increased proliferation of pathogenic bacteria in both humans and animals, and thus to bacterial infections, particularly in farm animals wherein the use of antibiotics has been widely decreasing.

The possibility of supplementing the diet, both in human subjects and in monogastric animals, with synthesis amino acids, i.e. amino acids of synthetic origin but identical to those present in natural sources of proteins, allows to finalise the fulfilment of nutritional requirements (limiting amino acids) without wasting proteins or excess undigested proteins in the gut.

The literature has shown that there are various problems in the administration of amino acids to human subjects or monogastric animals. In particular, free amino acids are strongly acidic, therefore, when administered through enteral route they can cause problems of heartburn or gastric ulcers. Furthermore, tryptophan degrades to acid pH like that of the stomach (pH 2-3), in particular under fasting. Therefore, there is a high demand to have amino acids in protected forms that allow their transit in the stomach both without causing damage to the walls of the gastric tract and without undergoing degradation.

In addition, in monogastric subjects (human or animal) with an intestinal epithelium damaged by stress (for example, stress caused by intensive animal farming) and by inflammatory insults of various nature (such as, harmful ingredients present in the diet, pathogenic infections, environmental stress such as heat stress, etc.) the action of efficient absorption of nutrients (for example amino acids) by the intestine is impaired, given that the intestinal epithelium is the area in which the enterocytes responsible for the absorption of nutrients reside.

Therefore, the technical problem addressed and solved by the present invention lies in providing compositions (pharmaceutical compositions, dietary supplements, food, feed, feed additives or nutraceutical compositions) suitable for providing components involved in protein synthesis, such as amino acids, proteins and/or Krebs cycle intermediates, to a monogastric subject, preferably a human subject or pig, to support the normal development of muscle mass or to favour the increase thereof, in particular in the case of amino acid deficiencies.

In addition, the technical problem addressed and solved by the present invention lies in providing said subject with said components involved in protein synthesis (for example, amino acids) so that the blood bioavailability thereof is constant within a period of time from 2 hours to 24 hours, in order to limit the fluctuations of the blood levels thereof in between the main meals.

Lastly, the technical problem addressed and solved by the present invention lies in providing said subject with said amino acids and/or gastroprotected proteins so that they can be administered through enteral route without causing damage to the walls of the gastric tract and/or without undergoing degradation in a strongly acidic environment.

Furthermore, besides amino acids as essential elements for their growth and development, the technical problem addressed and solved by the present invention lies in providing said monogastric subjects (such as humans or pigs), also with compounds capable of maintaining and/or restoring the integrity and homeostasis of the intestinal epithelium, so as to ensure efficient and effective absorption of the amino acids, less waste of energy by the body, and an effective and rapid response by the immune system. The primary function of the intestine lies in an efficient digestion and absorption of nutrients and these functions are affected if there are stress conditions that damage the intestinal mucosa, creating malabsorption and/or limiting the development of an immune response in case of need Thus, it is possible to find an inadequate or unbalanced amino acid supply quality/quantity-wise in subjects having a damaged intestinal epithelium as a result of stresses induced at bowel level (such as bacterial infections, parasitic infections, environmental stresses, etc.).

After an intense research and development phase, the Applicant found a composition (in short composition of the invention), comprising both a first active component, such as at least one amino acid and/or at least one whey protein, and a second active component, such as at least one protease, alpha-ketoglutaric acid, ornithine, a phytocompound derivative or a mixture thereof, wherein said first and second active component are embedded or incorporated into/by a controlled-release lipid matrix, which meets the requirements of adequate supply of amino acids and/or proteins to a monogastric subject, preferably human or pig, with the blood bioavailability of said amino acids constant over the 24 hours following the oral administration of said composition to said subject.

The combination of the composition of the invention with the diet of the subject allows to increase the efficiency of the amino acids and/or proteins administered and, thus, to decrease the % of protein in the diet of the subject, leading to an economic advantage and to decrease the nitrogen excreted hence limiting the environmental impact in the case of monogastric farm animals, preferably pigs.

Furthermore, the presence of Krebs cycle intermediates, such as alpha-ketoglutarate and/or ornithine in the composition of the invention facilitates the disposal of ammonia, produced by the catabolism of amino acids and highly toxic, stimulating the urea cycle. The urea cycle occurs in the liver and it is aimed at transforming ammonia into urea. Proteases (or proteolytic enzymes) are enzymes that enhance protein digestion at intestinal level. The presence of protease in the composition of the invention allows to stimulate the digestion of protein portions that arrive undigested in the intestine, contributing toward increasing the amino acid pool at the intestinal level and preventing the undigested protein portions from being used in the intestine as a substrate by bacterial pathogens.

The presence of phytocompound derivatives in the composition of the invention guarantees the supply—to said monogastric subjects (such as humans or pigs)—of substances with antioxidant, anti-inflammatory and/or immunostimulant properties for the maintenance and/or restoration of the integrity of the intestinal mucosa. In addition, said phytocompound derivatives described in the present invention show an antibacterial activity which contributes to intestinal health. Providing both an adequate supply of specific amino acids known for their trophic effect on the intestinal mucosa, and phytocompound derivatives capable of maintaining and/or restoring the state of health of the intestinal mucosa and treating the dysbiosis of the intestinal epithelium, helps to compensate for malabsorption of nutrients (for example amino acids) due to direct stress on the mucosa, and it helps the production of key proteins for an effective immune response.

In addition, the controlled-release lipid matrix of the invention allows gastroprotection and controlled release of the amino acids and other active components present in the composition of the invention into the intestine, ensuring their constant blood bioavailability over a period of time comprised from 2 hours to 24 hours. In the embodiment in which besides the amino acids the lipid matrix also embeds said phytocompound derivatives, said lipid matrix, disintegrating along the intestinal tract slowly and progressively (within a period of time comprised from 30 minutes to 8 hours, preferably 1 hour to 6 hours), allows the active components embedded therein to be gradually released into the various portions of the gastrointestinal tract. A gradual release of said phytocompound derivatives into the various portions of the gastrointestinal tract improves the effectiveness of the antioxidant, anti-inflammatory, immunostimulant and antibacterial activity thereof.

Furthermore, the compositions of the invention are devoid of adverse effects and, therefore, can be administered to a wide range of human subjects, including paediatric subjects, the elderly, and pregnant women. Lastly, the compositions of the invention are easy to prepare and cost-effective.

These and other objects, which will be clear from the detailed description that follows, are achieved by the composition and by the mixture of the present invention thanks to the technical characteristics claimed in the attached claims.

### DETAILED DESCRIPTION OF THE INVENTION

Forming an object of the present invention is a composition (according to a first embodiment (FR-I) or a first embodiment (FR-II)), comprising (i) a mixture of active components (in short, mixture of the invention) comprising or, alternatively, consisting of: (a) at least one first active component selected from the group comprising, or alternatively, consisting of (a1) at least one amino acid, or an acceptable pharmaceutical or food grade salt thereof, (a2) at least one whey protein and mixtures thereof; and (b) at least one second active component selected from the group comprising, or alternatively, consisting of (b1) at least one protease, (b2) alpha-ketoglutaric acid, (b3) ornithine, (b4) at least one phytocompound derivative botanical) (such as, thymol, carvacrol, eugenol, capsaicin, tannins, verbascoside and mixtures thereof) and a mixture thereof; wherein said composition further comprises (ii) a controlled release lipid matrix embedding or incorporating or dispersing said (i) mixture of active components (in short, lipid matrix of the invention), wherein said (ii) controlled release lipid matrix comprises or, alternatively, consists of at least one saturated or unsaturated, free or esterified fatty acid having a number of carbon atoms comprised in the range of C10-C30, preferably C14-C24, and/or at least one triglyceride having saturated or unsaturated fatty acid chains, having a number of carbons comprised in the range C6-C30, preferably C14-C24, and/or at least one wax having a number of carbon atoms comprised in the range C16-C36, preferably C24-C36; and, optionally, said composition comprises (iii) at least one an acceptable pharmaceutical or food grade additive and/or excipient; wherein, when said composition is administered through oral route, said (ii) lipid matrix allows gastroprotection and a controlled release of the active components of said (i) mixture (i. e. (a1), (a2 or derivative thereof), (b1), (b2), (b4) and/or (b3) and, optionally (c)) into the intestine, ensuring a constant blood bioavailability of the administered amino acids within a period of time comprised in the range from 2 hours to 24 hours (or 18 hours).

According to said first embodiment (FR-I) the composition of the invention comprises said (i) mixture of active components comprising, or alternatively, consisting of: said (a) at least one first active component selected from (a1) at least one amino acid, (a2) at least one whey protein and mixtures thereof; and said (b) second active component selected from (b1), (b2), (b3) and mixtures thereof; wherein said composition further comprises said (ii) controlled release lipid matrix, and, optionally, said (iii) at least one additive and/or excipient.

According to said second embodiment (FR-II) the composition of the invention comprises said (i) mixture of active components comprising, or alternatively, consisting of: said (a) at least one first active component selected from (a1) at least one amino acid, (a2) at least one whey protein and mixtures thereof; and said (b4) at least one phytocompound derivative (botanical) (such as, thymol, carvacrol, eugenol, capsaicin, tannins, verbascoside and mixtures thereof); wherein said composition further comprises said (ii) controlled release lipid matrix, and, optionally, said (iii) at least one additive and/or excipient

Preferably, according to said embodiment FR-II, the composition of the invention is a composition, preferably a solid composition in the form of granules, comprising (i) a mixture of active components comprising or, alternatively, consisting of: said (a.1) at least one amino acid, or a salt thereof, selected from a group comprising or, alternatively, consisting of: lysine (Lys), methionine (Met), tryptophan (Trp), leucine (Leu), valine (Val), isoleucine (ISO-Leu), phenylalanine and mixtures thereof; and said (b.4) at least one phytocompound derivative (botanical) selected from a group comprising or, alternatively, consisting of: thymol, carvacrol, eugenol, capsaicin, tannins, verbascoside and mixtures thereof; and wherein said composition further comprises said (ii) lipid matrix comprising or, alternatively, consisting of: at least one saturated or unsaturated, free or esterified fatty acid having a number of carbon atoms comprised in the range C10-C30, and/or at least one triglyceride having saturated or unsaturated fatty acid chains, having a number of carbons comprised in the range C6-C30, and/or at least one wax having a number of carbon atoms comprised in the range C16-C36; and optionally, said (iii) at least one additive and/or excipient. Examples of said embodiment FR-II are shown below (from FR-II-1 to FR-II-10).

Preferably, in the composition of the invention (according to FR-I or FR-II) said (a) first active component (i.e. (a1) and/or (a2)) and said (b) second active component (i.e. (b1) and/or (b2) and/or (b3) and/or (b4)) are at an ((a):(b) weight ratio in the range 1:10 to 10 preferably at a by weight ratio with respect to each other comprised in the range from 1:10 to 10 preferably from 1:5 to 5:1, more preferably from 1:3 to 3:1, even more preferably 1:1. For example, the [(a1) at least one amino acid: (b4) at least one phytocompound derivative] by weight ratio may be comprised in the range from 1:10 to 10:1, preferably from 10:1 to 10:5, more preferably from 10:1 to 10:3.

According to an aspect of said first embodiment (FR-I) of the composition of the invention (comprising (i) and (ii) and, optionally, (iii)), said (i) comprises (a1) at least one amino acid, wherein (a1) is selected from the group A comprising or, alternatively, consisting of: histidine, isoleucine, leucine, lysine, methionine, phenylalanine, threonine, tryptophan, valine, arginine, cysteine, tryptophan and glutamine; preferably glutamine, phenylalanine, lysine, methionine, threonine, tryptophan, valine, isoleucine and/or leucine; more preferably leucine, valine and isoleucine; even more preferably leucine; and said (b) is selected from (b1), (b2) and (b3), preferably (b) is (b2). When the composition of the invention (i.e. (i), (ii) and, optionally, (iii)) is intended for a human subject, preferably said (a1) is leucine and said (b) is selected from (b1), (b2) and (b3), preferably (b) is (b2) alpha-ketoglutaric acid. When the composition of the invention (i.e. (i), (ii) and, optionally, (iii)) is intended for a pig, preferably said (a1) is selected from lysine, methionine, threonine, tryptophan and valine, and said (b)is selected from (b1), (b2) and (b3), preferably (b) is (b2) alpha-ketoglutaric acid.

According to an aspect of said embodiment FR-I, said (a1) at least one amino acid is not lysine and/or tryptophan.

According to an aspect of said embodiment FR-I, said (a1) at least one amino acid is not tryptophan when the composition of the invention comprises sulfamethazine or sulfadimidine (SMT) (I UPAC name 4-amino-N-(4,6-dimethylpyrimidin-2-yl) benzenesulfonamide, CAS N° 57-68-1).

According to an aspect of said embodiment FR-I, said (a1) at least one amino acid is not tryptophan when the (ii) lipid matrix of the invention comprises or, alternatively, consists of long-chain fatty acids, preferably a mixture of stearic acid, palmitic acid, oleic acid and myristic acid.

According to an aspect of said embodiment FR-I of the composition of the invention (comprising (i), (ii) and, optionally, (iii)), said (a1) at least one amino acid is a mixture of amino acids selected from the group B of mixtures comprising or, alternatively, consisting of: (B.1) leucine, valine and isoleucine (BCAAs); (B.2) leucine and at least one or more amino acids selected from the group A, preferably one or more selected from lysine, methionine, threonine, tryptophan, valine, isoleucine, histidine and glutamine, such as for example leucine and lysine, leucine and methionine, leucine and threonine, leucine and tryptophan, leucine and valine, leucine and isoleucine, leucine and histidine, leucine and glutamine; leucine and lysine and one selected from methionine, threonine, tryptophan, valine, isoleucine, histidine and glutamine; leucine and methionine and one selected from lysine, threonine, tryptophan, valine, isoleucine, histidine and glutamine; leucine and threonine and one selected from lysine, methionine, tryptophan, valine, isoleucine, histidine and glutamine; leucine and tryptophan and one selected from lysine, methionine, threonine, valine, isoleucine, histidine and glutamine; leucine and valine and one selected from lysine, methionine, threonine, tryptophan, isoleucine, histidine and glutamine; leucine and isoleucine and one selected from lysine, methionine, threonine, tryptophan, valine, histidine and glutamine; leucine and histidine and one selected from lysine, methionine, threonine, tryptophan, valine, isoleucine and glutamine; leucine and glutamine and one selected from lysine, methionine, threonine, tryptophan, valine, isoleucine and histidine; leucine and isoleucine and valine and one selected from lysine, methionine, threonine, tryptophan, histidine and glutamine; (B.3) leucine, isoleucine, valine, lysine, methionine, threonine and tryptophan; (B.4) leucine, isoleucine, valine, lysine, methionine, threonine, tryptophan and histidine; (B.5) lysine, methionine, threonine, tryptophan; (B.6) lysine, methionine, threonine, tryptophan and valine; and said (b) at least one second active component is selected from the group comprising or, alternatively, consisting of (b1) at least one protease, (b2) alpha-ketoglutaric acid, (b3) ornithine, and a mixture thereof; preferably (b) is (b2) alpha-ketoglutaric acid.

Preferably, when said subject is a human subject, said (a1) mixture of amino acids selected from said group B is (B.1), such as a mixture of leucine, isoleucine and valine (BCAAs), and said (b) is selected from (b1), (b2) and (b3), preferably (b) is (b2) alpha-ketoglutaric acid. Preferably, when said subject is a human subject, said (a1) mixture of amino acids selected from said group B is selected from (B.2) and (b) is selected from (b1), (b2) and (b3), preferably (b) is (b2) alpha-ketoglutaric acid. Preferably, when said subject is a human subject, said (a1) is (B.3) and (b) is selected from (b1), (b2) and (b3), preferably (b) is (b2) alpha-ketoglutaric acid. Preferably, when said subject is a human subject, said (a1) is (B.4) and (b) is selected from (b1), (b2) and (b3), preferably (b) is (b2) alpha-ketoglutaric acid. Preferably, when said subject is a pig, said (a1) is (B.5) and (b) is selected from (b1), (b2) and (b3), preferably (b) is (b2) alpha-ketoglutaric acid. Preferably, when said subject is a pig, said (a1) is (B.6) and (b) is selected from (b1), (b2) and (b3), preferably (b) is (b2) alpha-ketoglutaric acid. Preferably, in said mixtures of 2 amino acids selected from said group (B.2), preferably leucine and lysine, leucine and methionine, leucine and threonine, leucine and tryptophan, leucine and valine, leucine and isoleucine, leucine and histidine, leucine and glutamine, the two amino acids are at a by weight ratio with respect to each other comprised in the range from 1:10 to 10:1, preferably from 1:5 to 5:1, more preferably from 1:3 to 3:1, even more preferably 1:1.

In the context of the present invention, the term “amino acids” refers to L- or D- α-amino acids, i.e. those whose amino group and carboxylic group are bound to the same carbon atom, called carbon α, in L or D configuration, thus having relative optical activity, with the sole exception of glycine, which is achiral. Amino acids are the constitutive units of proteins (proteinogenic); a huge number of proteins can be obtained depending on the type, number and sequence order with which the different amino acids bind. In nature, we are generally familiar with 20 proteinogenic amino acids. The organism of a monogastric subject can synthesise some of the amino acids required to build proteins, but it is not capable of building others, which are therefore called “essential amino acids” (EAAs) and must be introduced through food.

“Whey protein” or whey proteins is a mixture of proteins isolated from cow whey, such as the liquid material which forms a cheese production by-product. Cow milk proteins consist of about 20% whey and 80% casein proteins, while the protein in breast milk is about 60% whey and 40% casein. Serum proteins are generally a mixture of β-lactoglobulins, α-lactoglobulins, serum albumins, and other minor fractions, which are soluble in their native form, irrespective of pH. The protein fraction in whey (about 10% of the dry matter in the serum) comprises four main protein fractions and six minor protein fractions. The main protein fractions of whey are: β-lactoglobulins (˜65%), α-lactoglobulins (˜25%), serum albumins (˜8%); whereas the minor fractions (˜2%) of whey are: lactoferrins, immunoglobulins, glycomacropeptides, lactoperoxidase, lysozyme. Furthermore, whey proteins consist of about 40-50% essential amino acids (EAAs) and they are considered a rich source of these amino acids. An example of the amino acid composition of whey proteins is reported below in Table 1.

TABLE 1

| | g/100 g of proteins of whey |
|---|---|
| Essential AAs (EAAs) | |
| Threonine | 5.4 |
| Methionine | 1.8 |
| Phenylalanine | 2.5 |
| Histidine | 1.4 |
| Lysine | 7.1 |
| Valine | 3.5 |
| Isoleucine | 3.8 |
| Leucine | 8.6 |
| Non-essential AAs (NEAAs) | |
| Serine | 4.0 |
| Glycine | 1.5 |
| Glutamic acid | 16.0 |
| Proline | 8.7 |
| Cysteine | 0.1 |
| Alanine | 2.6 |
| Tyrosine | 4.4 |
| Arginine | 2.9 |

In the context of the present invention, the term “phytocompound derivative (botanical)” is used to indicate a chemical compound naturally present in plants as a secondary metabolite, which can also be chemically synthesised in a laboratory. When extracted from plants, it can be obtained as a powder extract of various parts of the plant such as stem, roots, seeds (dry extract), or as a liquid extract (essential oils) or semi-solid extract (oleoresins). Said phytocompound derivatives (from (a) to (g)) are prepared according to methods and apparatus known to the man skilled in the art, such as for example aqueous or hydroalcoholic extractions from plants (dry extract) or from essential oils, distillations, enzymatic or microbiological processes from material of plant origin in the raw state or after the transformation thereof (by drying, roasting, fermentation, etc.), or chemical synthesis.

Thymol (IUPAC name: 2-isopropyl-5-methylphenol or 3-Hydroxy-4-isopropyltoluene) is a monoterpenoid phenol present in abundant quantities in plants of the genus Thymus, from which it takes its name and to which it contributes to the creation of aroma, together with other molecules such as carvacrol.

Carvacrol (IUPAC name 5-isopropyl-2-methylphenol) or cymophenol is a monoterpenoid phenol (regioisomer of thymol) present in the essential oil of oregano and thymus.

Eugenol (IUPAC name 2-methoxy-4-(prop-2-en-1-yl)-phenol) is a hydroxylated aromatic compound which is extracted from some essential oils, in particular clove oil and from cinnamon.

Capsaicin (IUPAC name (E)-N-(4-hydroxy-3-methoxybenzyI)-8-methylnon-6-enamide) (also called capsicine or capseicin) is an alkaloid present, at various concentrations, in plants of the genus Capsicum (for example in hot chili pepper).

Tannins are a class of compounds (polyphenols) contained in various plants with properties similar to those of tannic acid (IUPAC name: 2,3-dihydroxy-5-({[(2R,3R,4S,5R,6R)-3,4,5,6-tetrakis({3,4-dihydroxy-5-[(3,4,5-trihydroxyphenyl)carbonyloxy]phenyl}carbonyloxy)oxan-2-yl]methoxy}carbonyl)phenyl 3,4,5-trihydroxybenzoate. Tannins are common in vascular plants the sweet chestnut (Castanea sativa) which contains, in the tissues thereof, about 7% of the total, being the richest in tannins. The families of plants most known for the presence of tannins are: Aceraceae, Actinidiaceae, Anacardiaceae, Bixaceae, Burseraceae, Combretaceae, Dipterocarpaceae, Ericaceae, Grossulariaceae, Myricaceae among dicotyledons and Najadaceae and Typhaceae among monocotyledons.

Verbascoside (molecular formula C₂₉H₃₀O₁₅) is a phenylpropanoid which can be obtained by extraction from plants of the order of the Lamiales (family of Scrophulariaceae, such as Verbasicum phlomoides and Verbasum mallophorum) but also in species of the orders of Asterales, Cucurbitales and Magnoliales, or produced in plant cell cultures of Leucosceptrum sp (Lamiaceae) and Syringa sp (Oleaceae).

Saponins (or saponosides) are terpene glycosides of plant origin. They are present in two main classes of the plant kingdom, the Magnoliopsida (dicotyledons) and Liliopsida (monocotyledons).

The presence of said (b.4) at least one phytocompound derivative in the composition of the invention (according to FR-II) confers to said composition the properties of preserving the integrity and homeostasis of the intestinal mucosa and of enhancing the restoration of intestinal integrity in the presence of damage to the intestine (for example stress-related damage or inflammatory insult).

“Triglycerides” (or triacylglycerols) are neutral esters of glycerol in which chains of three long-chain fatty acids are present instead of the hydrogen atoms of the hydroxyl groups. The length of the fatty acid chains in the common triglycerides structures may be from 5 to 28 carbon atoms, but 17 and 19 are more common.

The term “fatty acids” (in short FAs) is mainly but not exclusively used to indicate long-chain aliphatic monocarboxylic acids (number of carbon atoms comprised in the range C10-C30) with an even number of carbon atoms, without branching and acyclic (i.e., consisting of molecules that do not have ring-like closed chains). Fatty acids can be saturated (if their molecule has single C—C bonds only) or unsaturated (if they have double C═C bonds).

The term “waxes” is used to indicate to long-chain fatty acid esters with high molecular weight monohydric alcohols. Waxes may be of plant origin or animal origin (beeswax). Beeswax consists of various compounds, including for example: hydrocarbons 14%, monoesters 35%, diesters 14%, triesters 3%, hydroxy monoesters 4%, hydroxy polyesters 8%, acid esters 1%, acidic polyesters 2%, free acids 12%, free alcohols 1%, not identified 6%. The main components of beeswax are palmitates, palmitic acid, hydroxypalmitates and oleate esters formed by long chains (30-32 carbon atoms) of aliphatic alcohols, with a 6:1 ratio between the two main components triacontanyl palmitate (myricyl palmitate) CH₃(CH₂)₂₉O—CO—(CH₂)14 CH₃ and cerotic acid CH₃(CH₂)₂₄COOH. Beeswax has a melting comprised between 62° C. and 64° C. Density at 15° C. ranges between 0.958 g/cm³ and 0.970 g/cm³. Beeswax can be classified into two broad categories: European type and Eastern type. The saponification number is 3-5 for European type and 8-9 for Eastern type.

Advantageously, said fatty acid comprised in the (ii) controlled-release lipid matrix (both in FR-I and FR-II) can be a hydrogenated or non-hydrogenated fatty acid, of plant and/or animal origin.

Advantageously, said triglyceride comprised in the (ii) controlled-release lipid matrix (both in FR-I and in FR-II) can be a hydrogenated or non-hydrogenated triglyceride, of plant and/or animal origin.

Advantageously, said waxes comprised in the (ii) controlled release lipid matrix (both in FR-I and FR-II) can be of plant and/or animal origin; preferably beeswax.

In a preferred embodiment, said (ii) controlled release lipid matrix (both in FR-I and FR-II) comprises or, alternatively, consists of at least one hydrogenated fatty acid of plant and/or animal origin and/or at least one hydrogenated triglyceride of plant and/or animal origin and/or at least one wax; preferably at least one hydrogenated fatty acid of plant origin and/or at least one hydrogenated triglyceride of plant origin and/or at least one wax of animal origin. Said at least one hydrogenated fatty acid of plant origin and/or said at least one hydrogenated triglyceride of plant origin and/or said at least one wax of animal origin are selected from the group comprising: palm oil, sunflower oil, maize oil, rapeseed oil, peanut oil, olive oil, soybean oil and beeswax; preferably palm oil, rapeseed oil, soybean oil and mixtures thereof. Triglycerides of animal origin are selected from: chicken fat, hydrogenated chicken fat, bovine tallow and pork lard, even in the hydrogenated form.

Rapeseed oil is a vegetable food oil produced from rape seed (Brassica napus, Brassica rapa, Brassica juncea) and from cultivars or mutant varieties. A rapeseed oil which can be used in the context of the invention has, for example, the following distribution of fatty acids: myristic acid <0.5%, palmitic acid 9-16%, stearic acid: 79-89%, oleic acid and its isomers <4%, linoleic acid and its isomers <1%, linolenic acid and its isomers <0.2%, arachidic acid <1%, behenic acid <1%, saturated fatty acids with carbon chain length less than C14: <0.1%.

Palm oil (or palm fruit oil and palm seed oil) are vegetable oils, mainly consisting of triglycerides with high concentrations of saturated fatty acids, obtained from oil palms, mainly Elaeis guineensis but also from Elaeis oleifera and Malea maripa. A palm oil which can be used in the context of the invention has, for example, the following distribution of fatty acids: lauric acid <1%, myristic acid <2%, palmitic acid <47%, stearic acid <58%, oleic acid <3%, trans fatty acids <1%.

Soy oil (or soy bean oil) is obtained by extraction from soy beans through a particular process called crush using chemical solvents. Soybean oil mainly consists of triglycerides with the following typical distribution of fatty acids, as indicated in Codex Alimentarius: palmitic acid 8.0-13.5%, stearic acid 2.0-5.4%, oleic acid 17-30%, linoleic acid 48.0-59.0%, α-linolenic acid 4.5-11.0%, and others up to 100%.

According to a preferred aspect, the composition of the invention according to the embodiment FR-I comprises: said (i) mixture of active components comprising (a1) at least one amino acid and/or (a2) at least one whey protein, and said (b) second active component selected from (b1), (b2), (b3) and mixtures thereof; wherein said composition further comprises said (ii) lipid matrix selected from, palm oil, rapeseed oil, soybean oil and mixtures thereof (preferably soybean oil) and, optionally, said (iii) at least one additive and/or excipient (preferably coating additives selected from group F).

According to a further preferred aspect, the composition of the invention according to embodiment FR-II comprises: said (i) mixture of active components comprising (a1) at least one amino acid and/or (a2) at least one whey protein (preferably (a1)), and said (b4) at least one phytocompound derivative (botanical) (such as thymol, carvacrol, eugenol, capsaicin, tannins, verbascoside and mixtures thereof);wherein said composition further comprises said (ii) lipid matrix selected from, palm oil, rapeseed oil, soybean oil and mixtures thereof (preferably soybean oil) and, optionally, said (iii) at least one additive and/or excipient (preferably coating additives selected from group F). For example, according to said embodiment FR-II, the composition of the invention is a composition, preferably a solid composition in the form of granules, comprising (i) a mixture of active components comprising, or alternatively, consisting of: said (a.1) at least one amino acid, or a salt thereof, selected from a group comprising, or alternatively, consisting of: lysine (Lys), methionine (Met), tryptophan (Trp), leucine (Leu), valine (Val), isoleucine (Iso-Leu), phenylalanine and mixtures thereof; and said (b.4) at least one phytocompound derivative (botanical) selected from a group comprising or, alternatively, consisting of: thymol, carvacrol, eugenol, capsaicin, tannins, verbascoside and mixtures thereof; and wherein said composition further comprises said (ii) lipid matrix selected from the group comprising or, alternatively, consisting of: palm oil, rapeseed oil, soybean oil and mixtures thereof (preferably soybean oil); and, optionally, said (iii) at least one additive and/or excipient (preferably coating additives selected from group F).

According to an aspect of said embodiment FR-I or FR-II, the composition of the invention—comprising (i), (ii) and, optionally, (iii)—comprises said (i) mixture of active components, comprising (a1), (a2), (b1), (b2), (b4) and/or (b3) and, optionally (c) according to any one of the embodiments of the invention, at a percentage by weight comprised in the range from 1% to 90% (for example 5%, 20%, 25%, 30%, 35%, 40%, 50%, 55% or 65%), with respect to the total weight of the composition, preferably from 5% or 10% to 50%, more preferably from 15% to 45%, and said (ii) controlled release lipid matrix, according to any one of the embodiments of the invention, at a % by weight comprised in the range from 10% to 80% (for example 15%, 20%, 25%, 35%, 50% or 65%), with respect to the total weight of the composition; preferably from 40% to 60% or from 30% to 70%, more preferably from 45% to 55%. Said % of (ii) represent the total % of (ii), irrespectively of the components comprised in (iii), for example comprising or not comprising (iii. 1).

According to an aspect of said second embodiment of the invention (FR-II, for example from FR-II-1 to FR-II-10), the composition of the invention comprises: said (i.1) at least one amino acid from 1% to 80% (for example 5%, 10%, 15%, 20%, 25% or 30%), preferably from 5% to 40%, more preferably from 5% to 35%, said (i.2) at least one phytocompound derivative from 0.5% to 15% (for example 5%, 10%, 15%, 20%, 25% or 30%), preferably from 1% to 10%, more preferably from 1% to 5%, said (ii) lipid matrix from 10% to 80% (for example 15%, 20%, 25%, 35%, 50% or 65%); preferably from 30% to 70%, more preferably from 45% to 55%, and, optionally, said (iii) additive and/or excipient (preferably said (iii.1) coating additive) from 0.1% to 30% (for example 0.5%, 2%, 4%, 6%, 8%, 15% or 25%), preferably from 1% to 20%, more preferably from 5% to 10%; wherein said percentages are percentages by weight with respect to the total weight of the composition.

According to a preferred aspect of the embodiment FR-I, the composition of the invention comprises:

(i) a mixture comprising, or alternatively, consisting of leucine, or an acceptable pharmaceutical or food grade salt thereof, and (b) at least one second active component selected from (b1), (b2), (b3) and mixtures thereof, preferably (b2) alpha-ketoglutaric acid, and, optionally, one or more amino acids selected from said group A or group B;
(ii) a controlled release lipid matrix as defined in the context of the present invention comprising or, alternatively, consisting of at least one at least one fatty acid and/or triglyceride and/or waxes or mixtures thereof, wherein said lipid matrix embeds or incorporates or disperses leucine and the other components comprised in the (i) mixture, conferring gastroprotection to the components comprised in the (i) mixture, a controlled release thereof into the intestine and a constant blood bioavailability thereof over a period of time comprised from 2 to 24 hours; and, optionally, said composition comprises (iii); wherein (i) and (ii) are present at a % by weight as defined in the present invention.

According to a further preferred aspect of the embodiment FR-I, the composition of the invention comprises:
(i) a mixture comprising or, alternatively, consisting of a<mixture of leucine and isoleucine and valine, or an acceptable pharmaceutical or food grade salt thereof, and (b) at least one second active component selected from (b1), (b2), (b3) and mixtures thereof, preferably (b2) alpha-ketoglutaric acid, and, optionally, one or more amino acids selected from said group A or group B;
(ii) a controlled release lipid matrix as defined in the context of the present invention comprising or, alternatively, consisting of at least one at least one fatty acid and/or triglyceride and/or waxes or mixtures thereof, wherein said lipid matrix embeds or incorporates or disperses the mixture of leucine, isoleucine and valine, and the other components comprised in the (i) mixture, conferring gastroprotection to the components comprised in the (i) mixture, a controlled release thereof into the intestine and a constant blood bioavailability thereof over a period of time comprised from 2 hours to 24 hours; and, optionally, said composition comprises (iii); wherein (i) and (ii) are present at a % by weight as defined in the present invention.

According to a further preferred aspect, the embodiment FR-I of the composition of the invention comprises:
(i) a mixture comprising, or alternatively, consisting of (a2) a whey protein and (b) at least one second active component selected from (b1), (b2), (b3) and mixtures thereof, preferably (b2) alpha-ketoglutaric acid, and, optionally, one or more amino acids selected from said group A or group B;
(ii) a controlled release lipid matrix as defined in the context of the present invention comprising or, alternatively, consisting of at least one at least one fatty acid and/or triglyceride and/or waxes or mixtures thereof, wherein said lipid matrix embeds or incorporates or disperses the components comprised in the (i) mixture, conferring gastroprotection to the components comprised in the (i) mixture, a controlled release thereof into the intestine and a constant blood bioavailability thereof over a period of time comprised from 2 hours to 24 hours; and, optionally, said composition comprises (iii); wherein (i) and (ii) are present at a % by weight as defined in the present invention.

The (i) mixture of active components embedded or incorporated or dispersed with/in said (ii) controlled release lipid matrix is produced through the production method described in patent document EP 1 391 155 A1 in paragraphs [0048]-[0049] and [0077] (in short, preparation method of the invention); said paragraphs are incorporated into the present description for reference. In short, said preparation method of the invention comprises the steps of:
step (I), preparing said (ii) controlled release lipid matrix according to any one of the embodiments of the invention and, if present, said (iii) at least one additive and/or excipient to obtain a homogeneous mass (I) (temperature about 80° C.-120° C.), followed by
step (II), dispersing in said homogeneous mass (I) said (i) mixture of active components (i.e. (a1), (a2), (b1), (b2), (b4) and/or (b3) and, optionally (c)), according to any of the embodiments of the invention, to obtain a mass (II) (temperature about 55° C.-70° C.), followed by
step (III) spray the mass (II) in a cold room (temperature lower than 15° C.) to obtain the composition of the invention, preferably in the form of substantially spherical particles, wherein the active components comprised in said (i) mixture (i.e. (a1), (a2), (b1), (b2), (b4) and/or (b3) and, optionally (c)) and, if present, said (iii) at least one additive and/or excipient are dispersed or embedded or incorporated by/in said (ii) controlled release lipid matrix.

In other words, the composition of the invention obtained by the preparation method of the invention is an aggregate of (a1), (a2), (b1), (b2), (b4) and/or (b3) and, optionally (c) and/or (ii) dispersed in said (ii) controlled release lipid matrix.

The term “(ii) lipid matrix of the invention embedding or incorporating or dispersing and/or microencapsulating said (i) mixture” does not identify active components comprised in the (i) mixture (for example (a1), (a2), (b1), (b2), (b4) and/or (b3) and, if present (c)) coated with a film of said (ii) controlled release lipid matrix. Furthermore, the term “(ii) lipid matrix of the invention embedding or incorporating or dispersing and/or microencapsulating said (i) mixture” does not identify active components comprised in the (i) mixture (for example (a1), (a2), (b1), (b2), (b4) and/or (b3) and, if present (c)) in the form of tablets, pills or analogues wherein said tablets, pills or analogues are coated with the (ii) controlled-release lipid matrix or with film of said (ii).

The advantages of the composition of the invention, in particular the long-term constant blood bioavailability (2 hours-24 hours) of the active components (i.e. amino acids) comprised in the (i) mixture of the invention, derive both from the chemical/physical properties of said (ii) controlled release lipid matrix (for example lipophilicity, resistance to disintegration at acid pH, rate of disintegration at neutral or slightly acid pH and in the presence of enzymes) and from the preparation method of the invention which allows to embed or disperse or incorporate the active components (a1), (a2), (b1), (b2), (b4) and/or (b3) and, if present (c), in the (ii) lipid matrix.

Said (ii) controlled release lipid matrix allows, after administration of the composition of the invention to a monogastric subject through oral route, a controlled release of the active components present in the (i) mixture (for example (a1), (a2), (b1), (b2), (b4) and/or (b3) into the intestine and, optionally (c)) as a function of time and digestive process. Therefore, said (ii) controlled-release lipid matrix is capable of guaranteeing a constant amount of the active components present in the (i) mixture of active components (i.e. amino acids) in the blood (or plasma) and, therefore, a constant blood bioavailability of said active components over 24 hours (over a period of time comprised in the range from 2 hours to 24 hours), advantageously limiting their fluctuations between the main meals.

In the context of the present invention, the term “bioavailability” is used to indicate the “absolute bioavailability”, such as a fraction of compound under analysis in the systemic circulation following non-intravenous (e.g. oral) administration. Absolute bioavailability compares the bioavailability of a compound in the systemic circulation following non-intravenous (e.g. oral) administration with the bioavailability of the same compound following intravenous administration. It is therefore the fraction of compound absorbed through non-intravenous administration compared with that of the corresponding intravenous administration. The comparison should be normalised with respect to the dose, therefore the absorbed amount is corrected by dividing by the corresponding administered dose. The absolute bioavailability for a compound administered through oral route (po) is obtained from the oral area under the curve (AUCₚₒ) corrected by dose (D) divided by the intravenous area under the curve (AUCᵢᵥ) [F_{abs}=100·(AUCₚₒ·Dᵢᵥ)/(AUCᵢᵥ·Dₚₒ) %]. Said bioavailability can be measured by means of methods and apparatus known to the man skilled in the art, for example according to the methodology reported hereinafter in the present experimental part.

According to a second definition, in the context of the present invention, the term “bioavailability” is used to indicate the “relative bioavailability”, such as fraction of a compound under analysis (e.g. compound according to the invention) in the systemic circulation following the oral administration thereof in comparison with the fraction of a comparison compound (e.g. a feed or a composition not according to the invention) in the systemic circulation following the oral administration thereof. Said relative bioavailability of the compound under analysis can be expressed as a percentage considering 100% the fraction absorbed in the blood of the comparison compound: in this case, the percentage expressing the relative bioavailability of the compound under analysis may be less than 100% (lower bioavailability with respect to the comparison compound) or higher than 100% (higher bioavailability with respect to the comparison compound). Alternatively, said relative bioavailability of the compound under analysis can be expressed as a percentage difference with respect to the 1 (or 100) value of the blood-absorbed fraction of the comparison compound. For example, the following method may be used to determine the bioavailability of a composition according to the invention comprising lysine and a phytocompound derivative and a lipid matrix (e.g. rapeseed oil): two animal study groups are prepared, group 1 is administered with 1 kg of feed containing 40% of proteins, of which said proteins contain 10% of lysine (1 kg feed=40 g of lysine); group 2 is administered with an amount of composition of the invention containing 40 g of lysine. At a set time, the blood is collected from the animals of group 1 and group 2 and the mean lysine value present in the blood (in short, amount of lysine) is determined (for example by HPLC-MS) for each group. The amount of lysine determined for group 1 is set as a value 1 or a value of 100%, the amount of lysine determined for group 2 is expressed as a percentage or percentage difference with reference to said value 1 or 100%. Thus, if the amount of lysine in the blood of the Group 2 animals is, for example, 1.2 μg/ml and the amount of lysine in the blood of the Group 1 animals is 1.0 μg/ml, the bioavailability (relative bioavailability) of lysine of the composition of the invention is 120% or 20% more with respect to the bioavailability of lysine administered through the feed.

Preferably, in the context of the present invention the bioavailability data are expressed as relative bioavailability given that quantification of amino acid levels in the blood by means of the absolute bioavailability method is difficult to apply.

The term “bioavailability of a constant amino acid” is used to indicate a constant value of relative bioavailability (with respect to the bioavailability of the same amino acid provided by a feed or a comparison compound) comprised from 101% to 200%, preferably from 101% to 150%, more preferably from 101% to 125%, equivalent to a positive percentage difference (greater bioavailability of the amino acid provided by the compound of the invention with respect to the feed or comparison) comprised from 1% to 100%, preferably from 1% to 50%, more preferably from 1% to 25% (for example 2%, 4%, 5%, 6%, 8%, 10%, 15%, 20%).

Furthermore, said (ii) controlled release lipid matrix provides gastroprotection of the active components comprised in the (i) mixture of the invention, given that said (ii) matrix is stable at the acid pH of the stomach (pH 2-3). Thus, said (ii) lipid matrix incorporating and/or embedding said active components comprised in the (i) mixture of the invention allows the transit thereof through the stomach without undergoing degradation and without the amino acids, acidic substances, causing damage to the walls of the gastric tract. When the composition of the invention reaches the intestine, where the pH has a higher value with respect to the stomach (pH 6-7.5) and where an enzymatic system capable of digesting the lipid compounds (i.e. lipase) is present, said (ii) lipid matrix dissolves slowly (over a period of time comprised from 30 minutes to 8 hours, preferably from 1 hour to 6 hours) allowing said controlled release of said active components (such as amino acids and phytocompound derivatives) at the intestinal level and, thus, a blood bioavailability of amino acids administered constantly over a period of time comprised from 2 hours to 24 hours. Furthermore, the intestine has an enzymatic system rich in lipases which, by digesting the lipid matrix, allow the controlled release of the active components.

To demonstrate the efficacy of the lipid matrix in the gastroprotection of active components comprised in a composition, there was conducted a study in which there was monitored the presence of sorbic acid and vanillin (markers) in the content of several sections of the gastrointestinal tract of a first group of pigs to which there had been orally administered a composition comprising natural acids, including sorbic acid, and flavours, including vanillin, encapsulated in a lipid matrix and a second group of pigs to which there were administered the same free components (not encapsulated in the lipid matrix). Said study shows that the two markers, sorbic acid and vanillin, are present in the various intestinal tracts only when administered in encapsulated form in the lipid matrix, given that this lipid matrix allows to bypass the stomach and allow a slow release at the intestinal level, where markers are absorbed and made available in the blood, resulting in increased blood bioavailability.

Furthermore, a study was conducted to demonstrate the prolonged bioavailability of the active ingredients following their encapsulation with a lipid matrix, using sulfamethazine as study marker. In particular, a composition comprising sulfamethazine encapsulated with a lipid matrix was administered to a first group of pigs through oral route and a composition comprising free sulfametazine (not encapsulated in a lipid matrix) was administered to a second group at a 1 g/pig dose. Eight hours after administration, the absorbed fraction of sulfamethazine embedded into the lipid matrix revealed to be lower than 31.8±13% with respect to sulfamethazine in free form. With the form encapsulated in the lipid matrix, the absorption of sulfamethazine was completed in 24 hours, while with the free form it was completed in 10 hours, highlighting the effect of controlled release over time and constant blood bioavailability over 24 hours for the form encapsulated with lipid matrix.

Sorbic acid, vanillin and sulfamethazine, were used as markers of lipid matrix release instead of the amino acids given that analytically it is challenging to determine—at the intestinal level—the presence of the limiting amino acids released from the compositions under analysis given the high content of amino acids from food, from intestinal desquamation cells and microbial proliferation.

In the context of the present invention, the term “subject” is used to indicate a monogastric human or animal subject, preferably a human subject (or human) or pig.

In the context of the present invention, the term “monogastric” is used to indicate an animal in which the stomach has only one compartment, inside which chemical and enzymatic digestion takes place. On the contrary, polygastric or ruminant animals have the stomach consisting of four different compartments: rumen, reticulum, omasum and the abomasum (which is the equivalent of the stomach of monogastric animals since the only one with gastric mucosa). This group includes Camelids (with a three-compartment stomach) and Ruminants in the strict sense (Bovids, Cervids, Giraffids, etc.). Polygastric animals have a better ability to digest plant foods due to rumination and microbial digestion, which takes place in rumen.

Preferably, the aggregate formed through the spray step (iii) of the preparation method of the invention, wherein the (i) mixture of active components (for example (a.1) amino acids and (b.4) phytocompound derivatives) is embedded or incorporated or dispersed in/with said (ii) controlled release lipid matrix, according to any one of the embodiments of the invention (FR-I or FR-II), is in the form of substantially spherical particles (synonyms: granules or microcapsules) having an average particle diameter (or particle size) comprised in the range from 50 μm to 2500 μm or 100 μm to 2000 μm (for example 250 μm-400 μm or 500 μm), preferably from 200 μm to 1500 μm or from 400-500 μm to 1500 μm, more preferably from 250 μm to 1000 μm. In particular, when the composition of the invention is intended for human subjects, said average particle diameter is preferably in the range from 100 μm to 1000 μm or higher like in the pig. On the other hand, when the composition of the invention is intended for pigs, said average particle diameter is preferably in the range of from 500 μm to 2000 μm, more preferably from 500 μm to 1500 μm or from 500 μm to 1000 μm.

Within a batch of composition of the invention (according to FR-I or FR-II) in the form of granules, the granules do not all have the same particle size, but they have a particle size distribution percentage within the above indicated particle size ranges. Said particle size distribution percentage may vary depending on whether the composition is for use in the treatment of an amino acid deficiency in human subjects or pigs.

For pigs or human subjects, a batch of 100 granules of the composition may have the following particle size distribution percentage: from 25% to 35% of granules has a particle size from 500 μm to 1000 μm, from 45% to 55% 1000 μm-1500 μm, from 20% to 30% 1500 μm-2000 μm, from 0.1% to 1% 2000 μm-2500 μm (wherein said percentages are percentages of granules with respect to 100 granules).

Examples of batches of the composition of the invention (according to FR-I or FR-II) in the form of granules are reported in Table A. Said particle size percentage distribution is constant and reproducible in the preparation of the batches of the composition of the invention. Upon reaching the intestine, said granules break up at different times and in different sections of the intestine depending on their granulometry. Thus, the effect of said particle size distribution percentage consists in a slow and progressive release of the active compounds embedded in the mixture M embedded in the lipid matrix along the whole section of the intestine. In particular, the smaller granules are digested (releasing the active ingredients) over a short period of time in the upper part of the intestine, whereas larger granules are digested by lipases more slowly and the release of active ingredients occurs over a longer period of time with respect to the smaller granules and more distal position along the intestinal tract.

TABLE A

| | | | | | | |
|---|---|---|---|---|---|---|
| 50 − 250 um | 250 ÷ 400 um | 400 ÷ 500 um | 500 ÷ 1000 um | 1000 ÷ 1500 um | 1500 ÷ 2000 um | 2000 − 2500 um |

| | | | | |
|---|---|---|---|---|
| 5% | 30% | 40% | 24.5% | 0.5% |

To define the particle size percentage distribution of a batch of the composition of the present invention, instruments and methodologies known to the man skilled in the art can be used for particle size analysis. For example, within the scope of the present invention, one of the following two methods can be used to define said particle size distribution percentage: particle size analysis using certified sieves or particle size analysis using laser diffraction.

The analysis by means of certified sieves (i.e. perforated plates made of stainless steel) is carried out, for example, by means of a vibrating platform with n sieves assembled one over the other in a sieve holder container arranged above the vibrating platform (for example, frequency of about 3000 cycles/min). Each sieve in the sieve holder container has a different size (for example sieves from 250 μm to 2500 μm) and said sieves are positioned one over the other so that the larger sieves are arranged in the upper part of the container and the smaller sieves in the lower part of the container. The container is vibrated and a certain amount of a powder or granules is poured onto the upper sieve: the particles passing through the upper sieves reach the lower sieves or beyond. The operation ends when no evident separation occurs anymore. Stopping the powder on a sieve of a certain size determines its particle size. The sieves are quality certified: the certificate of conformity certifies that the mesh, materials used, dimensions and production process comply with the requirements.

The analysis of the size of solid particles—using the laser diffraction technique—is based on the principle that the particles illuminated by a laser beam diffuse the light at an angle related to the size thereof (the angle increases as the particle size decreases). The average diameter is determined based on the surface/volume ratio, using the parameter D (De Brouckere mean diameter—equation). The dimensional distribution is identified by the following parameters: D (0.1), D (0.5), D (0.9), which represent the cumulative distribution diameters of 10%, 50% and 90% of the total particles.

In an embodiment of the composition of the invention, comprising (i), (ii) and, optionally, (iii) according to any one of the embodiments of the invention (according to FR-I or FR-II), besides at least one first active component (a1) and/or (a2) and at least one second active component selected from (b1), (b2), (b3), (b4) and mixtures thereof, the (i) mixture of the invention further comprises (c) at least one third non-amino acid active component selected from group C comprising or, alternatively, consisting of organic or inorganic acids, aromatic components, vitamins, mineral salts, antioxidants, probiotic bacterial strains, prebiotics and enzymes. (c), if present, being a component of the (i) mixture of the invention, also (c) is embedded or incorporated or dispersed by/in said (ii) controlled release lipid matrix.

Said vitamin is a vitamin of group A, B, C, D, E or K; preferably a vitamin of group B selected from the group comprising or, alternatively, consisting of B1, B2, B3, B4, B5, B6, B7, B8, B9, B10, B11, B12 and mixtures thereof.

Advantageously, said mineral salt is an organic or inorganic salt of a metal cation, such as, for example, Fe, Se, Mg, Ca, K, Zn, Cu.

Advantageously, said antioxidant is selected from N-acetyl cysteine (NAC), Coenzyme Q10 (CoQ10), acetyl-L-carnitine, and analogues.

Preferably, the weight ratio of said first active components (a1) and/or (a2) toward second active components (b1) and/or (b2) and/or (b3) and/or (b4) toward third non-amino acid active components (c) ((a):(b):(c)) is comprised in the range from 1:10:10 to 10:10:1 or 10:1:10, preferably from 1:5:5 to 5:5:1 or 5:1:5, more preferably from 1:3:3 to 3:3:1 or 3:1:3, even more preferably 1:1:1.

The composition of the invention, according to any one of the embodiments of the invention, may further comprise (iii.1) one or more coating additives. Said (iii.1) one or more coating additives are selected from the group comprising or, alternatively, consisting of: fumed silica, calcium stearate, magnesium stearate, calcium sulfate, precipitated silica, calcium silicate, aluminium silicate, hydrophobic silica and mixtures thereof; preferably fumed silica, calcium sulfate, precipitated silica, calcium silicate, aluminium silicate, hydrophobic silica and mixtures thereof. The (iii.1) one or more coating additives used are used to increase the viscosity of the matrix and decrease its permeability. Preferably, the composition of the invention comprises a plurality of said (iii.1) coating additives at a % by weight comprised in the range from 0.1% to 30% (for example 0.5%, 2%, 4%, 6%, 8%, 15% or 25%) with respect to the total weight of the composition, preferably between 1% to 20%, more preferably between 5% to 10%.

For example, the composition of the invention may comprise (according to FR-II): said (i) mixture of active components comprising or, alternatively, consisting of at least one at least one amino acid and at least one phytocompound derivative, said (ii) controlled release lipid matrix and, optionally, said (iii.1) at least one coating additive, wherein said composition comprises said (i), (ii) and, optionally, (iii.1) in the following percentages by weight with respect to the total weight of the composition: said (i) from 1% to 90% or 89.9% (for example 5%, 20%, 25%, 30%, 35%, 40%, 50%, 55% or 65%), said (ii) from 10% to 80% (for example 15%, 20%, 25%, 35%, 50% or 65%) and said (iii.1) from 0.1% to 30%; preferably said (i) from 5% to 50%, said (ii) from 30% to 70%, and said (iii.1) from 1% to 20%; more preferably said (i) from 15% to 40%, said (ii) from 40% to 60%, and said (iii.1) from 5% to 10%,

In the composition of the invention, comprising (i) and (ii) according to any one of the embodiments of the invention (FR-I or FR-II) and, said (iii) at least one pharmaceutical or food grade additive and/or excipient is a substance devoid of therapeutic activity suitable for pharmaceutical or food use. In the context of the present invention the acceptable ingredients for pharmaceutical or food use comprise all ancillary substances known to the man skilled in the art such as, by way of non-limiting example, diluents, solvents (including water, glycerine, ethyl alcohol), solubilisers, thickeners, sweeteners, flavourings, dyes, lubricants, surfactants, antimicrobials, antioxidants, preservatives, pH stabilisation buffers and the mixtures thereof. Non-limiting examples of such substances are maltodextrins, phosphate buffers, bases such as sodium hydroxide, xanthan gum, guar gum, fructose, natural or artificial flavours.

The composition of the invention, comprising (i) and (ii) and, optionally, (iii) and/or (iii.1) according to any one of the embodiments of the invention, may be a pharmaceutical composition, nutraceutical composition, dietary supplement product or food product or a food for special medical purpose, feed, feed additive or medical device composition.

In the context of the present invention, the expression “medical device” is used in the meaning according to the Italian Legislative Decree n° 46 dated 24 Feb. 1997 or according to the new Medical Device Regulation (EU) 2017/745 (MDR).

The composition of the present invention may be in a liquid form, such as solution, two-phase liquid system, suspension or syrup, semi-solid form, such as gel, cream or foam, or solid form, such as powder, granules, flakes, aggregates, capsules, pills, bars and equivalent forms.

Preferably, the composition of the invention is for oral (enteral) use, preferably in solid form of granules, microgranules, flakes or powder, for example microcapsules to be inserted into capsules or microgranules to be swallowed, to be inserted into supplements for humans and animals or to be inserted into complete food for humans and animals, or, alternatively, in suspension liquid form, for example granules, microgranules or powder in suspension.

When the composition of the invention is in the form of a tablet, it means that the aggregate formed between the active components comprised in the (i) mixture (i.e. (a) and/or (b) and, optionally, (c)) and the (ii) lipid matrix embedding or incorporating said active components, is processed to form a tablet.

The composition of the invention in tablet form is not a tablet coated with the (ii) lipid matrix of the invention.

Forming an object of the present invention is a composition of the invention, comprising said (i) and (ii) and, optionally, (ii) according to any one of the embodiments of the invention (FR-I or FR-II), obtained/obtainable according to the preparation method of the present invention (step (I), (II) and (III)) described above.

Forming an object of the invention is a composition of the invention, comprising (i) and (ii) and, optionally, (iii) according to any one of the embodiments of the invention (FR-I or FR-II), for use as a medicament.

The composition of the invention, comprising (i) and (ii) and, optionally, (iii) according to any one of the embodiments of the invention (FR-I or FR-II), is for use in a method for the treatment of an amino acid deficiency, wherein said method provides for supplying said amino acids to a monogastric subject, preferably a human subject or a pig.

The term “supply of amino acids” is used to indicate the average daily supply of amino acids (or proteins or analogues thereof) for the normal development of the muscle mass of the subject or for a greater or faster development of muscle mass with respect to the average development of the species to which the subject belongs.

The composition of the invention, comprising (i) and (ii) and, optionally, (iii) according to any one of the embodiments of the invention (FR-I or FR-II), is for use in a method for preventive and/or curative treatment of, a protein (or amino acid) deficiency and of a disease, symptom and/or disorder related with to said protein deficiency, in a subject in need.

Mild protein deficiency can cause: decreased metabolic efficiency (for example, ease of bleeding, slow wound healing, etc.), decrease in corpusculated elements in the blood, weight loss (as a result of muscle decrease, decreased muscle volume, early fatigue, difficulty in concentrating and learning, mood, muscle and/or joint and/or bone pain, glycemic changes, increased susceptibility to infection. Less frequently, mild protein deficiency can also cause: anxiety (due to the altered synthesis of neurotransmitters), decreased athletic performance (decreased compensation of the training stimulus), sleep alterations (some hypothesise that it may be caused by the alteration of tryptophan and serotonin synthesis), digestive deficiency (proteins allow the natural synthesis of digestive enzymes). In addition, a protein deficiency can generate more serious symptoms or disorders or diseases, such as muscle depletion (consisting of the auto-digestion of muscle proteins to produce energy), decreased muscle mass and strength and severe decrease in all the body's protein-based components such as nails, hair, skin, enzymes, neurotransmitters, hormones, immunoglobulins.

The composition of the invention, comprising (i) and (ii) and, optionally, (iii) according to any one of the embodiments of the invention (FR-I o FR-II), is for use in a method for preventive and/or curative treatment of a decreased muscle mass and/or decreased muscle strength and of a disease, symptom and/or disorder related with said decrease in muscle mass and/or decrease in muscle strength, for example sarcopaenia, muscle atrophy, muscular dystrophy, muscle catabolism, in a subject in need.

The present description also relates to a method for the preventive and/or curative treatment of an amino acid supply or a protein (or amino acid) deficiency or a decrease in muscle mass and/or muscle strength and of diseases, symptoms and/or disorders related therewith, wherein said treatment comprises the administration of a therapeutically effective amount of the composition of the invention as defined above (FR-I or FR-II) to a monogastric subject in need, preferably human or pig.

The expression “treatment method” in the context of the present invention is used to indicate an action, comprising the administration of a substance, or mixture of substances or combination thereof, with the aim of eliminating, reducing/decreasing or preventing a pathology or disease and its symptoms or disorders.

The term “therapeutically effective amount” refers to the amount of composition or mixture of active components that elicits the biological or medicinal response in a tissue, system, mammal, or human being that is sought and defined by an individual, researcher, veterinarian, physician, or other clinician or health worker.

Lastly, forming an object of the present invention is the use of the composition of the invention, comprising (i) and (ii) and, optionally, (iii) according to any of the embodiments of the invention (FR-I or FR-II), for the preparation of a feed or feed additive for a monogastric animal, preferably the pig.

Unless specified otherwise, the expression “composition or mixture comprising a component at an amount comprised in a range from x to y” is used to indicate that said component can be present in the composition or mixture at all the amounts present in said range, even though not specified, extremes of the range comprised.

In preferred examples of the composition of the invention according to said second embodiment (FR-II), said composition comprises: said (i) mixture of active components, said (ii) lipid matrix comprises or, alternatively, consists of: a triglyceride, a fatty acid, a wax and a mixture thereof (as defined in the present invention), and, optionally, said (iii) at least one additive and/or excipient,

wherein said (i) mixture of active components (comprising or, alternatively, consisting of (a.1) at least one amino acid and (b.4) at least one phytocompound derivative) is selected from the group comprising or, alternatively, consisting of:

FR-II-1: lysine and thymol, lysine and carvacrol, lysine and eugenol, lysine and capsaicin, lysine and tannins, lysine and verbascoside,

lysine and thymol and carvacrol, lysine and thymol and eugenol, lysine and thymol and capsaicin, lysine and thymol and tannins, lysine and thymol and verbascoside,

lysine and carvacrol and eugenol, lysine and carvacrol and capsaicin, lysine and carvacrol and tannins, lysine and carvacrol and verbascoside;

FR-II-2: methionine and thymol, methionine and carvacrol, methionine and eugenol, methionine and capsaicin, methionine and tannins, methionine mabd verbascoside,

methionine and thymol and carvacrol, methionine and thymol and eugenol, methionine and thymol and capsaicin, methionine and thymol and tannins, methionine and thymol and verbascoside,

methionine and carvacrol and eugenol, methionine and carvacrol and capsaicin, methionine and carvacrol and tannins, methionine and carvacrol and verbascoside;

FR-II-3: tryptophan and thymol, tryptophan and carvacrol, tryptophan and eugenol, tryptophan and capsaicin, tryptophan and tannins, tryptophan and verbascoside,

tryptophan and thymol and carvacrol, tryptophan and thymol and eugenol, tryptophan and thymol and capsaicin, tryptophan and thymol and tannins, tryptophan and thymol and verbascoside,

tryptophan and carvacrol and eugenol, tryptophan and carvacrol and capsaicin, tryptophan and carvacrol and tannins, tryptophan and carvacrol and verbascoside;

FR-II-4: leucine and thymol, leucine and carvacrol, leucine and eugenol, leucine and capsaicin, leucine and tannins, leucine and verbascoside,

leucine and thymol and carvacrol, leucine and thymol and eugenol, leucine and thymol and capsaicin, leucine and thymol and tannins, leucine and thymol and verbascoside,

leucine and carvacrol and eugenol, leucine and carvacrol and capsaicin, leucine and carvacrol and tannins, leucine and carvacrol and verbascoside;

FR-II-5: lysine and methionine and thymol, lysine and methionine and carvacrol, lysine and methionine and eugenol, lysine and methionine and capsaicin, lysine and methionine and tannins, lysine and methionine and verbascoside,

lysine and methionine and thymol and carvacrol, lysine and methionine and thymol and eugenol, lysine and methionine and thymol and capsaicin, lysine and methionine and thymol and tannins, lysine and methionine and thymol and verbascoside,

lysine and methionine and carvacrol and eugenol, lysine and methionine and carvacrol and capsaicin, lysine and methionine and carvacrol and tannins, lysine and methionine and carvacrol and verbascoside;

FR-II-6: lysine and methionine and tryptophan and thymol, lysine and methionine and tryptophan and carvacrol, lysine and methionine and tryptophan and eugenol, lysine and methionine and tryptophan and capsaicin, lysine and methionine and tryptophan and tannins, lysine and methionine and tryptophan and verbascoside,

lysine and methionine and tryptophan and thymol and carvacrol, lysine and methionine and tryptophan and thymol and eugenol, lysine and methionine and tryptophan and thymol and capsaicin, lysine and methionine and tryptophan and thymol and tannins, lysine and methionine and tryptophan and thymol and verbascoside,

lysine and methionine and tryptophan and carvacrol and eugenol, lysine and methionine and tryptophan and carvacrol and capsaicin, lysine and methionine and tryptophan and carvacrol and tannins, lysine and methionine and tryptophan and carvacrol and verbascoside,

FR-II-7: lysine and methionine and leucine and thymol, lysine and methionine and leucine and carvacrol, lysine and methionine and leucine and eugenol, lysine and methionine and leucine and capsaicin, lysine and methionine and leucine and tannins, lysine and methionine and leucine and verbascoside,

lysine and methionine and leucine and thymol and carvacrol, lysine and methionine and leucine and thymol and eugenol, lysine and methionine and leucine and thymol and capsaicin, lysine and methionine and leucine and thymol and tannins, lysine and methionine and leucine and thymol and verbascoside,

lysine and methionine and leucine and carvacrol and eugenol, lysine and methionine and leucine and carvacrol and capsaicin, lysine and methionine and leucine and carvacrol and tannins, lysine and methionine and leucine and carvacrol and verbascoside,

FR-II-8: lysine and tryptophan and thymol, lysine and tryptophan and carvacrol, lysine and tryptophan and eugenol, lysine and tryptophan and capsaicin, lysine and tryptophan and tannins, lysine and tryptophan and verbascoside, lysine and tryptophan and saponins, lysine and tryptophan and thymol and carvacrol;

methionine and tryptophan and thymol, methionine and tryptophan and carvacrol, methionine and tryptophan and eugenol, methionine and tryptophan and capsaicin, methionine and tryptophan and tannins, methionine and tryptophan and verbascoside, methionine and tryptophan and saponins, methionine and tryptophan and carvacrol;

lysine and leucine and thymol, lysine and leucine and carvacrol, lysine and leucine and eugenol, lysine and leucine and capsaicin, lysine and leucine and tannins, lysine and leucine and verbascoside, lysine and leucine and thymol and carvacrol;

methionine and leucine and thymol, methionine and leucine and carvacrol, methionine and leucine and eugenol, methionine and leucine and capsaicin, methionine and leucine and tannins, methionine and leucine and verbascoside, methionine and leucine and thymol and carvacrol;

tryptophan and leucine and thymol, tryptophan and leucine and carvacrol, tryptophan and leucine and eugenol, tryptophan and leucine and capsaicin, tryptophan and leucine and tannins, tryptophan and leucine and verbascoside, tryptophan and leucine and thymol and carvacrol;

FR-II-9: lysine and thymol and valine and/or isoleucine, lysine and carvacrol and valine and/or isoleucine, lysine and histidine and eugenol, lysine and capsaicin and valine and/or isoleucine, lysine and tannins and valine and/or isoleucine, lysine and verbascoside and valine and/or isoleucine, lysine and thymol and carvacrol and valine and/or isoleucine;

methionine and thymol and valine and/or isoleucine, methionine and carvacrol and valine and/or isoleucine, methionine and histidine and eugenol, methionine and capsaicin and valine and/or isoleucine, methionine and tannins and valine and/or isoleucine, methionine and verbascoside and valine and/or isoleucine, methionine and thymol and carvacrol and valine and/or isoleucine;

tryptophan and thymol and valine and/or isoleucine, tryptophan and carvacrol and valine and/or isoleucine, tryptophan and eugenol and valine and/or isoleucine, tryptophan and capsaicin and valine and/or isoleucine, tryptophan and tannins and valine and/or isoleucine, tryptophan and verbascoside and valine and/or isoleucine, tryptophan and thymol and carvacrol and valine and/or isoleucine;

leucine and thymol and valine and/or isoleucine, leucine and carvacrol and valine and/or isoleucine, leucine and eugenol and valine and/or isoleucine, leucine and capsaicin and valine and/or isoleucine, leucine and tannins and valine and/or isoleucine, leucine and verbascoside and valine and/or isoleucine, leucine and thymol and carvacrol and valine and/or isoleucine;

lysine and methionine and thymol and valine and/or isoleucine, lysine and methionine and carvacrol and valine and/or isoleucine, lysine and methionine and eugenol and valine and/or isoleucine, lysine and methionine and capsaicin and valine and/or isoleucine, lysine and methionine and tannins and valine and/or isoleucine, lysine and methionine and verbascoside and valine and/or isoleucine, lysine and methionine and thymol and carvacrol and valine and/or isoleucine;

FR-II-10: lysine and leucine and valine and isoleucine and thymol, lysine and leucine and valine and isoleucine and carvacrol, lysine and leucine and valine and isoleucine and eugenol, lysine and leucine and valine and isoleucine and capsaicin, lysine and leucine and valine and isoleucine and tannins, lysine and leucine and valine and isoleucine and verbascoside, lysine and leucine and valine and isoleucine and thymol and carvacrol;

methionine and leucine and valine and isoleucine and thymol, methionine and leucine and valine and isoleucine and carvacrol, methionine and leucine and valine and isoleucine and eugenol, methionine and leucine and valine and isoleucine and capsaicin, methionine and leucine and valine and isoleucine and tannins, methionine and leucine and valine and isoleucine and verbascoside, methionine and leucine and valine and isoleucine and thymol and carvacrol;

tryptophan and leucine and valine and isoleucine and thymol, tryptophan and leucine and valine and isoleucine and carvacrol, tryptophan and leucine and valine and isoleucine and eugenol, tryptophan and leucine and valine and isoleucine and capsaicin, tryptophan and leucine and valine and isoleucine and tannins, tryptophan and leucine and valine and isoleucine and verbascoside, tryptophan and leucine and valine and isoleucine and thymol and carvacrol;

leucine and valine and isoleucine and thymol, leucine and valine and isoleucine and carvacrol, leucine and valine and isoleucine and eugenol, leucine and valine and isoleucine and capsaicin, leucine and valine and isoleucine and tannins, leucine and valine and isoleucine and verbascoside, leucine and valine and isoleucine and thymol and carvacrol;

lysine and methionine and leucine and valine and isoleucine and thymol, lysine and methionine and leucine and valine and isoleucine and carvacrol, lysine and methionine and leucine and valine and isoleucine and eugenol, lysine and methionine and leucine and valine and isoleucine and capsaicin, lysine and methionine and leucine and valine and isoleucine and tannins, lysine and methionine and leucine and valine and isoleucine and verbascoside, lysine and methionine and leucine and valine and isoleucine and thymol and carvacrol.

In preferred examples of the composition of the invention according to FR-II, said composition comprises: said (i) mixture of active components, said (ii) lipid matrix comprises or, alternatively, consists of rapeseed oil and, optionally, said (iii) at least one additive and/or excipient (preferably (iii.1) coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

In preferred examples of the composition of the invention according to FR-II, said composition comprises: said (i) mixture of active components, said (ii) lipid matrix comprises or, alternatively, consists of palm oil and, optionally, said (iii) at least one additive and/or excipient (preferably (iii.1) coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

In preferred examples of the composition of the invention according to FR-II, said composition comprises: said (i) mixture of active components, said (ii) lipid matrix comprises or, alternatively, consists of soybean oil and, optionally, said (iii) at least one additive and/or excipient (preferably (iii.1) coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

Preferred aspects)(FR-I-n°) of said first embodiment of the invention (FR-I) are reported below:

FR-I-1. A composition comprising

(i) a mixture of active components comprising, or alternatively, consisting of
(a) at least one first active component selected from the group comprising or, alternatively, consisting of:
(a1) at least one amino acid, or an acceptable pharmaceutical or food grade salt thereof,
(a2) a milk whey protein, and the mixtures thereof; and
(b) at least one second active component selected from the group comprising or, alternatively, consisting of:
(b1) at least one protease,
(b2) alpha ketoglutaric acid,
(b3) ornithine, and the mixtures thereof;

wherein said composition further comprises

(ii) a controlled release lipid matrix embedding or incorporating said (i) mixture of active components, wherein said (ii) controlled release lipid matrix comprises or, alternatively, consists of a saturated or unsaturated, free or esterified fatty acid having a number of carbon atoms comprised in the range C10-C30, and/or at least one triglyceride having saturated or unsaturated fatty acid chains, having a number of carbon atoms comprised in the range C6-C30 and/or at least one wax having a number of carbon atoms comprised in the range C16-C36; and optionally, said composition comprises

(iii) at least one acceptable pharmaceutical or food grade additive and/or excipient;

wherein said (ii) lipid matrix provides gastroprotection and a controlled release of the active components comprised in said (i) mixture in the intestine, guaranteeing a constant blood bioavailability thereof over a period of time comprised in the range from 2 hours and 24 hours.

FR-I-2. The composition according to FR-I-1, wherein said (a1) at least one amino acid is selected from the group A comprising or, alternatively, consisting of: histidine, isoleucine, leucine, lysine, methionine, phenylalanine, threonine, tryptophan, valine, arginine, cysteine, glutamine and mixtures thereof.

FR-I-3. The composition according to FR-I-1 or FR-I-2, wherein said (a1) at least one amino acid is a mixture of leucine and at least one or more amino acids selected from the group comprising or, alternatively, consisting of: lysine, methionine, threonine, tryptophan, valine, isoleucine, histidine and glutamine.

FR-I-4. The composition according to any one of the preceding FRs-I, wherein said (a1) at least one amino acid is leucine or a mixture of leucine, valine and isoleucine; and said (b) at least one second active component is (b2) alpha-ketoglutaric acid.

FR-I-5. The composition according to any one of the preceding FRs-I, wherein said (i) mixture of active components further comprises (c) at least one third non-amino acid active component selected from the group C comprising or, alternatively, consisting of: at least one vitamin, preferably a vitamin of group B, at least one organic or inorganic acid, at least one mineral salt, preferably an organic or inorganic salt of an Fe, Se, Mg, Ca, K, Zn or Cu cation, at least one antioxidant, at least one probiotic bacterial strain, at least one prebiotic, at least one enzyme and mixtures thereof.

FR-I-6. The composition according to any one of the preceding FRs-I, wherein said (ii) controlled release lipid matrix further comprises (iii.1) at least one coating additive selected from the group comprising or, alternatively consisting of: fumed silica, calcium stearate, magnesium stearate, calcium sulfate, precipitated silica, calcium silicate, aluminium silicate, hydrophobic silica.

FR-I-7. The composition according to any one of FRs-I from 1 to 6, wherein said composition is for use in a method for the treatment of amino acid supply in a monogastric subject, preferably a human subject or a pig.

FR-I-8. The composition according to any one of FRs-I from 1 to 6, wherein said composition is for use in a method for the treatment of protein deficiency, or of a disease, symptom and/or disorder related with said protein deficiency, in a monogastric subject in need, preferably a human subject or a pig.

FR-I-9. The composition according to any one of FRs-I from 1 to 6, wherein said composition is for use in a preventive and/or curative treatment of a decrease in muscle mass and/or decrease in muscle strength and of a disease, symptom and/or disorder related with said decrease in muscle mass and/or strength, in a monogastric subject in need; preferably for use in a method for the treatment of sarcopaenia, muscle atrophy, muscular dystrophy, muscle catabolism.

FR-I-10. Use of the composition according to any one of FRs-I from 1 to 6 for preparing an animal feed or feed additive for a monogastric animal, preferably a pig.

Experimental Part I

A method for measuring the plasma bioavailability of amino acids in a mammalian monogastric animal (for example the pig) following the administration of a composition according to the present invention (comprising at least one amino acid, at least one phytocompound and a lipid matrix) consists of:
administer the following diets to 3 experimental groups of an animal species under study (for example, chicken or fish):

group 1. a control diet (for example soy-based),

group 2. a diet added with a comparison composition: composition comprising amino acids and phytocompound derivatives in the absence of a lipid matrix (non-embedded active components), and group 3. a diet added with a composition according to the invention: composition comprising amino acids and phytocompound derivatives in the presence of a lipid matrix (embedded active components)
collect blood samples from the animals under study and obtain the plasma fraction. The samples are collected at different time-points after the administration of the diets (from 10 minutes up to 360 minutes after the administration) and the presence of one or more amino acids in the obtained plasma fractions is evaluated by means of the LC/MS-MS (Liquid Chromatography with tandem Mass Spectrometry) plasma amino acid assay method.

Experimental Part II

Table 2 shows the values of the experimental study which analysed the release of phytocompound derivatives embedded in a lipid matrix in the form of granules (composition according to the invention). As the data show, the release is a function of the time and size of the granules, the larger the granule size, the slower the release of the active ingredient.

The data were obtained by incubating 1 gram of granules of different sizes in a buffer simulating the intestinal pH conditions. At each time point (1 h, 2 h, 4 h) the phytocompound derivatives still present in the granules were quantified (by means of HPLC), and the release percentages were calculated by difference. The experiment was triplicated.

TABLE 2

| | Fraction at | Fraction at | Fraction at |
|---|---|---|---|
| Particle size | 1 hour | 2 hours | 3 hours |
| 2000-2500 um | 0 | 0 | 0 |
| 1500-2000 um | 0 | 17% | 8% |
| 1000-1500 um | 0 | 18% | 3% |
| 500-1000 um | 0 | 15% | 20% |
| 50-500 um | 26% | 36% | 48% |

### EXAMPLES

Representative examples of compositions of the invention according to the second embodiment (FR-II) are shown in Table 3

TABLE 3

| | | | | | | | | | Matrix oil |
|---|---|---|---|---|---|---|---|---|---|
| | AA1 (%) | AA2 (%) | AA3 (%) | der-FT1(%) | | der-FT2(%) | | Add (%) | (%) |
| Comp 1 | Lys | Met | — | a | (5%) | b | (2%) | (5%) | rapeseed |
| | (18%) | (15%) | | | | | | | (55%) |
| Comp 2 | Lys | Met | Thr | a | (10%) | c | (5%) | (3%) | rapeseed |
| | (10%) | (5%) | (5%) | | | | | | (62%) |
| Comp 3 | Lys | Val | IsoLeu | b | (12%) | c | (2%) | (1%) | palm |
| | (15%) | (10%) | (10%) | | | | | | (50%) |
| Comp 4 | Thr | Met | Trp | b | (4%) | d | (10%) | (3%) | palm |
| | (10%) | (5%) | (8%) | | | | | | (60%) |
| Comp 5 | Thr | Val | IsoLeu | d | (10%) | g | (6%) | (10%) | soy |
| | (10%) | (5%) | (5%) | | | | | | (54%) |
| Comp 6 | Leu | Met | — | c | (15%) | d | (2%) | (3%) | soy |
| | (10%) | (15%) | | | | | | | (55%) |
| Comp 7 | Lys | Met | — | a | (8%) | e | (2%) | (15%) | rapeseed |
| | (15%) | (15%) | | | | | | | (45%) |

(%): weight/weight composition. AA: Amino acid. der-FT: phytocompound derivative [(a) thymol, (b) carvacrol, (c) eugenol, (d) capsaicin, (e) tannins, (f) verbascoside]. Add: additive.

1. A solid composition in the form of granules for treatment of deficiency of at least one amino acid in a mammalian monogastric subject,
wherein said mammalian monogastric subject is a human or a pig, and
wherein said composition comprises
a mixture of active components comprising
at least one amino acid or an acceptable pharmaceutical or food grade salt thereof, wherein said amino acid is selected from the group consisting of lysine, methionine, tryptophan, leucine, valine, isoleucine, phenylalanine and mixtures thereof; and
at least one derivative of a phytocompound selected from the group consisting of thymol, carvacrol, eugenol, capsaicin, tannins, verbascoside and mixtures thereof;
wherein said composition further comprises
a lipid matrix embedding said mixture of active components, wherein said lipid matrix comprises
at least one saturated or unsaturated, free or esterified fatty acid having a number of carbon atoms in the range of C10-C30, and/or
at least one triglyceride having saturated or unsaturated fatty acid chains, having a number of carbons in the range of C6-C30 and/or
at least one wax having a number of carbon atoms in the range of C16-C36;
wherein said composition in the form of granules has the following particle size distribution percentage with respect to 100 granules: from 5% to 10% of granules having an average particle size from 50 μm to 500 μm, from 25% to 35% of granules having an average particle size from 500 μm to 1000 μm, from 45% to 55% of granules having an average particle size from 1000 μm to 1500 μm, from 20% to 30% of granules having an average particle size from 1500 μm to 2000 μm, or from 0.1% to 1% of granules having an average particle size of from 2000 μm to 2500 μm;
wherein said composition is formulated for administration through oral route,
wherein said lipid matrix is capable of providing gastroprotection of said at least one amino acid and said at least one phytocompound derivative,
wherein said lipid matrix is capable of providing a controlled release of said at least one amino acid and said at least one phytocompound derivative within a time range from 30 minutes to 8 hours in the intestinal tract, and
wherein said lipid matrix is capable of providing a blood bioavailability of said at least one amino acid in a constant percentage over a period of time from 2 hours to 24 hours.

2. The composition according to claim 1, wherein said lipid matrix is selected from the consisting of rapeseed oil, palm oil, soybean oil and a mixture thereof.

3. The composition according to claim 1,
wherein said mixture of active components comprises lysine and said at least one phytocompound derivative, and
wherein said lipid matrix comprises rapeseed oil or palm oil or soybean oil or a mixture thereof.

4. The composition according to claim 1,
wherein said mixture of active components comprises methionine and said at least one phytocompound derivative, and
wherein said lipid matrix comprises rapeseed oil or palm oil or soybean oil or a mixture thereof.

5. The composition according to claim 1,
wherein said mixture of active components comprises tryptophan and said at least one phytocompound derivative, and
wherein said lipid matrix comprises rapeseed oil or palm oil or soybean oil or a mixture thereof.

6. The composition according to claim 1,
wherein said mixture of active components comprises leucine and said at least one phytocompound derivative, and
wherein said lipid matrix comprises rapeseed oil or palm oil or soybean oil or a mixture thereof.

7. The composition according to claim 1,
wherein said mixture of active components comprises lysine and methionine and said at least one phytocompound derivative, and
wherein said lipid matrix comprises rapeseed oil or palm oil or soybean oil or a mixture thereof.

8. The composition according to claim 1,
wherein said mixture of active components comprises lysine and methionine and tryptophan and said at least one phytocompound derivative, and
wherein said lipid matrix comprises rapeseed oil or palm oil or soybean oil or a mixture thereof.

9. The composition according to claim 1,
wherein said mixture of active components comprises lysine and methionine and leucine and said at least one phytocompound derivative, and
wherein said lipid matrix comprises rapeseed oil or palm oil or soybean oil or a mixture thereof.

10. The composition according to claim 1,
wherein said mixture of active components is selected from the group consisting of
lysine and tryptophan and thymol, lysine and tryptophan and carvacrol, lysine and tryptophan and eugenol, lysine and tryptophan and capsaicin, lysine and tryptophan and tannins, lysine and tryptophan and verbascoside, lysine and tryptophan and saponins, lysine and tryptophan and thymol and carvacrol;
methionine and tryptophan and thymol, methionine and tryptophan and carvacrol, methionine and tryptophan and eugenol, methionine and tryptophan and capsaicin, methionine and tryptophan and tannins, methionine and tryptophan and verbascoside, methionine and tryptophan and saponins, methionine and tryptophan and carvacrol;
lysine and leucine and thymol, lysine and leucine and carvacrol, lysine and leucine and eugenol, lysine and leucine and capsaicin, lysine and leucine and tannins, lysine and leucine and verbascoside, lysine and leucine and thymol and carvacrol;
methionine and leucine and thymol, methionine and leucine and carvacrol, methionine and leucine and eugenol, methionine and leucine and capsaicin, methionine and leucine and tannins, methionine and leucine and verbascoside, methionine and leucine and thymol and carvacrol;
tryptophan and leucine and thymol, tryptophan and leucine and carvacrol, tryptophan and leucine and eugenol, tryptophan and leucine and capsaicin, tryptophan and leucine and tannins, tryptophan and leucine and verbascoside, tryptophan and leucine and thymol and carvacrol; and
wherein said lipid matrix comprises rapeseed oil or palm oil or soybean oil or a mixture thereof.

11. The composition according to claim 1,
wherein said mixture of active components is selected from the group consisting of
lysine and thymol and valine and/or isoleucine, lysine and carvacrol and valine and/or isoleucine, lysine and histidine and eugenol, lysine and capsaicin and valine and/or isoleucine, lysine and tannins and valine and/or isoleucine, lysine and verbascoside and valine and/or isoleucine, lysine and thymol and carvacrol and valine and/or isoleucine;
methionine and thymol and valine and/or isoleucine, methionine and carvacrol and valine and/or isoleucine, methionine and histidine and eugenol, methionine and capsaicin and valine and/or isoleucine, methionine and tannins and valine and/or isoleucine, methionine and verbascoside and valine and/or isoleucine, methionine and thymol and carvacrol and valine and/or isoleucine;
tryptophan and thymol and valine and/or isoleucine, tryptophan and carvacrol and valine and/or isoleucine, tryptophan and eugenol and valine and/or isoleucine, tryptophan and capsaicin and valine and/or isoleucine, tryptophan and tannins and valine and/or isoleucine, tryptophan and verbascoside and valine and/or isoleucine, tryptophan and thymol and carvacrol and valine and/or isoleucine;
leucine and thymol and valine and/or isoleucine, leucine and carvacrol and valine and/or isoleucine, leucine and eugenol and valine and/or isoleucine, leucine and capsaicin and valine and/or isoleucine, leucine and tannins and valine and/or isoleucine, leucine and verbascoside and valine and/or isoleucine, leucine and thymol and carvacrol and valine and/or isoleucine;
lysine and methionine and thymol and valine and/or isoleucine, lysine and methionine and carvacrol and valine and/or isoleucine, lysine and methionine and eugenol and valine and/or isoleucine, lysine and methionine and capsaicin and valine and/or isoleucine, lysine and methionine and tannins and valine and/or isoleucine, lysine and methionine and verbascoside and valine and/or isoleucine, lysine and methionine and thymol and carvacrol and valine and/or isoleucine; and
wherein said lipid matrix comprises rapeseed oil or palm oil or soybean oil or a mixture thereof.

12. The composition according to claim 1,
wherein said mixture of active components is selected from the group consisting of
lysine and leucine and valine and isoleucine and thymol, lysine and leucine and valine and isoleucine and carvacrol, lysine and leucine and valine and isoleucine and eugenol, lysine and leucine and valine and isoleucine and capsaicin, lysine and leucine and valine and isoleucine and tannins, lysine and leucine and valine and isoleucine and verbascoside, lysine and leucine and valine and isoleucine and thymol and carvacrol;
methionine and leucine and valine and isoleucine and thymol, methionine and leucine and valine and isoleucine and carvacrol, methionine and leucine and valine and isoleucine and eugenol, methionine and leucine and valine and isoleucine and capsaicin, methionine and leucine and valine and isoleucine and tannins, methionine and leucine and valine and isoleucine and verbascoside, methionine and leucine and valine and isoleucine and thymol and carvacrol;
tryptophan and leucine and valine and isoleucine and thymol, tryptophan and leucine and valine and isoleucine and carvacrol, tryptophan and leucine and valine and isoleucine and eugenol, tryptophan and leucine and valine and isoleucine and capsaicin, tryptophan and leucine and valine and isoleucine and tannins, tryptophan and leucine and valine and isoleucine and verbascoside, tryptophan and leucine and valine and isoleucine and thymol and carvacrol;
leucine and valine and isoleucine and thymol, leucine and valine and isoleucine and carvacrol, leucine and valine and isoleucine and eugenol, leucine and valine and isoleucine and capsaicin, leucine and valine and isoleucine and tannins, leucine and valine and isoleucine and verbascoside, leucine and valine and isoleucine and thymol and carvacrol;
lysine and methionine and leucine and valine and isoleucine and thymol, lysine and methionine and leucine and valine and isoleucine and carvacrol, lysine and methionine and leucine and valine and isoleucine and eugenol, lysine and methionine and leucine and valine and isoleucine and capsaicin, lysine and methionine and leucine and valine and isoleucine and tannins, lysine and methionine and leucine and valine and isoleucine and verbascoside, lysine and methionine and leucine and valine and isoleucine and thymol and carvacrol;
wherein said lipid matrix comprises rapeseed oil or palm oil or soybean oil or a mixture thereof.

13. The composition according to claim 1, wherein said composition further comprises at least one acceptable pharmaceutical or food grade additive and/or excipient, wherein said at least one additive and/or excipient comprises at least one coating additive selected from the group consisting of fumed silica, calcium stearate, magnesium stearate, calcium sulfate, precipitated silica, calcium silicate, aluminium silicate, hydrophobic silica.

14. The composition according to claim 1, wherein said composition comprises at a percentage by weight with respect to the total weight of the composition:
said mixture of active components from 5% to 50%, said [[(ii)]] lipid matrix from 30% to 70%.

15. A method for the preventive and/or curative treatment of a decrease in muscle mass and/or decrease in muscle strength and of a disease, symptom and/or disorder related with said decrease in muscle mass and/or muscle strength, the method comprising
administering to the mammalian monogastric subject a composition according to claim 1.

16. A feed or feed additive for a mammalian monogastric subject comprising a composition according to claim 1, wherein said mammalian monogastric subject is a human subject or a pig.

17. The composition according to claim 1, wherein said mixture of active components is selected from the group consisting of:
lysine and thymol, lysine and carvacrol, lysine and eugenol, lysine and capsaicin, lysine and tannins, lysine and verbascoside, lysine and thymol and carvacrol, lysine and thymol and eugenol, lysine and thymol and capsaicin, lysine and thymol and tannins, lysine and thymol and verbascoside, lysine and carvacrol and eugenol, lysine and carvacrol and capsaicin, lysine and carvacrol and tannins, lysine and carvacrol and verbascoside.

18. The composition according to claim 1, wherein said mixture of active components is selected from the group consisting of:
methionine and thymol, methionine and carvacrol, methionine and eugenol, methionine and capsaicin, methionine and tannins, methionine and verbascoside, methionine and thymol and carvacrol, methionine and thymol and eugenol, methionine and thymol and capsaicin, methionine and thymol and tannins, methionine and thymol and verbascoside, methionine and carvacrol and eugenol, methionine and carvacrol and capsaicin, methionine and carvacrol and tannins, methionine and carvacrol and verbascoside.

19. The composition according to claim 13, wherein said coating additive is comprised at a percentage by weight with respect to the total weight of the composition in a range from 1% to 20%.

20. A method for the preventive and/or curative treatment of sarcopaenia, muscle atrophy, muscular dystrophy, muscle catabolism, the method comprising administering to the mammalian monogastric subject a composition according to claim 1.
